(19) United States
(12) Patent Application Publication
(10) Pub. No.: US 2011/0086911 A1
(43) Pub. Date: Apr. 14, 2011

(54) NOVEL BISMUTH(III) NSAID COMPOUNDS AND METHODS FOR THEIR USE

(75) Inventors: Philip Craig ANDREWS, Caulfield South VIC (AU); Peter Courtney JUNK, Port Melbourne VIC (AU); Ish KUMAR, Glen Waverly VIC (AU); Richard L. FERRERO, Hawthorn East VIC (AU)

(73) Assignee: Monash University, Clayton (AU)

(21) Appl. No.: 12/578,118

(22) Filed: Oct. 13, 2009

(51) Int. Cl. A61K 31/29; C07F 9/94; A61P 31/04

(52) U.S. Cl. 514/503; 556/78

(57) ABSTRACT

The present invention relates to the field of non-specific anti inflammatory drugs (NSAIDs). In particular, the invention relates to bismuth(III) tris-carboxylate complexes having the formula [Bi(III)L₃]ₙ including its pharmaceutically acceptable salts and solvates, wherein, L is chosen from the group comprising carboxylato-NSAIDs, their derivatives, prodrugs or metabolytes, and n is ≧1.

The bismuth(III) tris-carboxylate complexes of the invention may be formulated for use in treatments for a wide range of ailments, particularly those where an anti-microbial activity is advantageous. In a particularly preferred embodiment the bismuth(III) tris-carboxylate complexes of the present invention exhibit activity against bacteria found in the gastrointestinal tract such as Helicobacter pylori, Escherichia coli, Klebsiella pneumoniae, Bacillus pumilus, Staphylococcus aureus and Staphylococcus epidermidis.

### FIELD OF INVENTION

The present invention relates to the field of non-specific anti inflammatory drugs (NSAIDs).

In one form, the invention relates to novel bismuth derivatives of NSAIDs having anti-microbial activity.

In one particular aspect the present invention is suitable for use minimising or inhibiting gastric injury and infection, particularly gastric injury caused by NSAIDs.

While it will be convenient to describe the invention in relation to antimicrobial activity against the bacteria Helicobacter pylori, it should be appreciated that the present invention is not so limited and may have antimicrobial activity against a wide range of other microbes found in, or on, a human or animal subject. Furthermore, while the antimicrobial activity of compounds according to the present invention may be directed to bacteria found in the gastrointestinal tract such as Helicobacter pylori, Escherichia coli, Klebsiella pneumoniae, Bacillus pumilus, Staphylococcus aureus and Staphylococcus epidermidis, again the invention is not so limited an may be directed to microbes found in, or on, other body organs. Accordingly, while the invention will be described with particular reference to ingestion via the gastrointestinal tract, it will be appreciated that the compounds of the present invention are not limited to oral administration, but can be administered by any convenient route including intravenous and topical administration.

### BACKGROUND ART

Throughout this specification the use of the word “inventor” in singular form may be taken as reference to one (singular) inventor or more than one (plural) inventor of the present invention.

It is to be appreciated that any discussion of documents, devices, acts or knowledge in this specification is included to explain the context of the present invention. Further, the discussion throughout this specification comes about due to the realisation of the inventor and/or the identification of certain related art problems by the inventor. Moreover, any discussion of material such as documents, devices, acts or knowledge in this specification is included to explain the context of the invention in terms of the inventor's knowledge and experience and, accordingly, any such discussion should not be taken as an admission that any of the material forms part of the prior art base or the common general knowledge in the relevant art in Australia, or elsewhere, on or before the priority date of the disclosure and claims herein.

### NSAIDs Generally

NSAIDs are among the most widely used and effective drugs. NSAIDs offer symptomatic relief for a large range of conditions by exerting anti-inflammatory, analgesic and anti-pyretic effects.⁸ Currently, there are approximately 50 variations available on the global market and many, such as ibuprofen and naproxen, are available as over-the-counter (non-prescription) medicines. However, there are health risks associated with their use, with the most common side effect being gastrointestinal (GI) damage: short to mid-term use can result in dyspepsia, diarrhoea, nausea and vomiting, while long term use can lead to severe bleeding, ulceration and even stomach cancer.^{9,10}

The GI damage caused by NSAIDs is related to the suppression of prostaglandin synthesis, through the inhibition of cyclo-oxygenase activity. Several strategies have been used to reduce the GI damage however none has yet been proven to have significant impact on the incidence of severe adverse reactions to NSAIDs.

The adverse effects of NSAIDs are sustained and amplified by the presence of Helicobacter pylori (H. pylori). H. pylori is a bacterium which causes gastritis and ulceration and has been implicated in the development of gastric cancer and gastric lymphoma. It is estimated that half the world's population is infected with H. pylori and without treatment the bacterium infects its host permanently. While antimicrobial regimens exist to treat the infection, these regimens are of limited usefulness due to the limited range of antibiotics that can effectively treat the infection, poor patient compliance and ever-increasing levels of H. pylori antibiotic resistance in the community. For example, in Australia, community H. pylori resistance rates for metronidazole and clarithromycin (two of the most commonly prescribed antibiotics) are 56% and 11% respectively.

The ability to minimise or inhibit gastric injury resulting from use of NSAIDs and microbial infection would be a major advance in patient care.

### Bismuth Compounds Generally

Bismuth compounds often exhibit good in-vitro and in-vivo activity as antimicrobial and anti-tumour agents' For example, when taken in conjunction with antibiotics such as tetracycline and metronidazole, bismuth subsalicylate (BSS), colloidal bismuth subcitrate (CBS) and ranitidine bismuth citrate (RBC) are effective in treating and eradicating H. pylori.¹⁻⁴ Treatments which include bismuth compounds offer two advantages; firstly they do not require a neutral stomach pH to be effective,⁵ and importantly there is no evidence of resistance as observed with standard antibiotic compounds.⁶ For example, the failure of triple therapies based on omeprazole (a proton pump inhibitor (PPI)), amoxicillin and clarithromycin in curing H. pylori infection is attributable to high rates of resistance to clarithromycin. In these instances quadruple rescue regimens are commonly invoked which involve one of the common bismuth drugs.⁶

Furthermore, bismuth(III) complexes derived from ciprofloxacin¹⁸ and norfloxacin¹⁹ show good antibacterial activity against Escherichia coli, Klebsiella pneumoniae, Bacillus pumilus, Staphylococcus aureus and Staphylococcus epidermidis. More recently it has been claimed that a 1:1 bismuth(III) complex derived from diclofenac, synthesised in situ from bismuth subcitrate and the Na salt of diclofenac, displayed an unusual increase in ulcerogenic activity in rats in comparison to the Na diclofenac salt itself, though the actual composition of the claimed 1:1 ‘bismuth diclofenac’ complex is not established.²⁰ However, a physical mixture of diclofenac and bismuth subcitrate did lead to a reduction in levels of ulceration when compared with diclofenac alone.

The scope for the continued development of new and effective bismuth based drugs is limited by the tendency of bismuth compounds to be hydrolytically sensitive, resulting in the formation and/or decomposition to oxide containing materials.¹¹ As a result pharmaceutical preparations of bismuth are often poorly defined and can suffer from a lack of stability, poor synthetic reproducibility, insolubility and limited characterisation.¹² These problems represent a clear impediment to the development of new bismuth-based drugs and define the challenge is preparing new compounds suitable for biological testing.

As a result there have been ongoing attempts to establish effective synthetic approaches, based on solvent free and solvent mediated methodologies, for the reproducible and high yielding formation of bismuth carboxylates.^{13,14} Some bismuth carboxylates have been characterised, including elucidation of their solid-state structures, and their stability and decomposition pathways studied.¹⁵ For example, the large salicylate encapsulated Bi₉ and Bi₃₈ oxo-clusters that constitute bismuth subsalicylate, have been formed on the slow hydrolysis in acetone of amorphous bismuth salicylate, [Bi(HSal)₃]ₙ.¹⁶ Salicylates constitute but one sub-class of NSAIDs, with three other sub-classes also based on carboxylic acids and accounting for more than half of those commonly available.

In view of the prominence of carboxylic acid based NSAIDs, there is a need for carboxylato-metal complexes, preferably bismuth(III) derived from commonly prescribed NSAIDs.

### SUMMARY OF INVENTION

An object of the present invention is to provide new NSAIDs or improved stability.

A further object of the present invention is to provide NSAIDs having anti-microbial indications.

Yet another object of the present invention is to inhibit or treat GI injury.

It is an object of the embodiments described herein to overcome or alleviate at least one of the above noted drawbacks of related art compounds or to at least provide a useful alternative to related art systems.

In a first aspect of embodiments described herein there is provided a bismuth(III) tris-carboxylate complex having the formula:

[Bi(III)L₃]ₙ

its pharmaceutically acceptable salts and solvates,
wherein,
L is chosen from the group comprising carboxylato-NSAIDs, their derivatives, prodrugs or metabolytes, and
n is ≧1.

Preferably the bismuth(III) tris-carboxylate complex of the present invention has anti-microbial activity, but may additionally or alternatively have other forms of biological activity.

In a preferred embodiment the bismuth(III) tris-carboxylate complex is a solvate of formula, [Bi(III) L₃Sₘ]ₙ wherein S is the solvate molecule, and m is 1, or ore preferably 1≦m≧3. For example, when the NSAID is ketoprofen or sulindac, the bismuth(III) tris-carboxylate complex may have the formula [Bi(III)L₃.(H₂O)]ₙ

Where used herein the term carboxylate or carboxylato refer to the moiety R—CO₂⁻ wherein R may be alkyl, aryl or combinations thereof and includes benzoates.

In a preferred embodiment the carboxylato-NSAID is chosen from the group comprising ketoprofen, naproxen, ibuprofen, 5-chlorosalicylic acid, mefenamic acid, diflunisal, fenbrufen, sulindac, probenecid, tolfenamic acid, flufenamic acid, meseclozone, their derivatives and metabolytes.

In a further preferred embodiment the metabolite is 5-chlorosalicylic acid, the main and active metabolite of the NSAID Meseclozone.¹⁷

The bismuth(III) tris-carboxylate complex of the present invention may be obtained in a crystalline form and/or in an amorphous form or as a mixture thereof. It is to be understood that all enantiomers and diasterioisomers and mixtures thereof are encompassed within the scope of the present invention.

In a second aspect of embodiments described herein there is provided a method of producing a bismuth(III) tris-carboxylate complex of formula:

[Bi(III)L₃]ₙ

its pharmaceutically acceptable salts and solvates,
wherein,
L is chosen from the group comprising carboxylato-NSAIDs their derivatives, prodrugs or metabolytes,
n is ≧1,
   the method comprising the step of reacting a Bi(III) compound with LH.

In a preferred embodiment the bismuth(III) compound is BiPh₃. Typically the ratio of Bi(III) to LH is 1:≧3.

The reaction may be solvent mediated or alternatively, solvent free.

Where it is desired to isolate the bismuth(III) compound as a salt, for example a pharmaceutically acceptable salt, this may be achieved by reacting the bismuth(III) compound in the form of the free base with an appropriate amount of LH, optionally in a suitable solvent (eg alcohol, ester or ether). Pharmaceutically acceptable salts may also be prepared from other salts using conventional methods.

The bismuth(III) compounds of the present invention may be isolated in association with solvent molecules by crystallisation from or evaporation of an appropriate solvent to give the corresponding solvates. For example, typically the solvent molecule will be small molecules such as H₂O, EtOH or MeOH. It will be appreciated that in many cases the solvate may be readily removed under vacuum, or by other means well known to the person skilled in the art. When a specific enantiomer of a bismuth(III) compound of the present invention is required, this may be obtained for example by resolution of a corresponding enantiomeric mixture using conventional methods such as, for example, HPLC.

In a third aspect of embodiments described herein there is provided a method of treating microbial infection including the step of administering to a subject a therapeutic amount of a bismuth(III) tris-carboxylate complex having the formula:

[Bi(III)L₃Sₘ]ₙ

wherein,
L is chosen from the group comprising carboxylato-NSAIDs their derivatives prodrugs or metabolites,
S is a solvent molecule,
0≦m≧3, and n is ≧1.
   In a preferred embodiment the carboxylato-NSAID is chosen from the group comprising;
ketoprofen (2-(3-benzoylphenyl)propanoic acid),
naproxen ((S)-2-(6-methoxynaphthalen-2-yl)propanoic acid),
ibuprofen ((S)-2-(4-isobutylphenyl)propanoic acid),
5-chlorosalicylic acid,
mefenamic acid (2-(2,3-dimethylphenylamino)benzoic acid),
diflunisal (2′,4′-difluoro-4-hydroxybiphenyl-3-carboxylic acid),
fenbrufen (γ-oxo-(1,1′-biphenyl)-4-butanoic acid),
sulindac ({(1Z)-5-fluoro-2-methyl-1-[4-(methylsulfinyl)benzylidene]-1H-indene-3-yl}acetic acid),
probenecid (4-(dipropylsulfamoyl)benzoic acid),
tolfenamic acid (2-[(3-chloro-2-methylphenyl)amino]benzoic acid)),
flufenamic acid (2-{[3-(trifluoromethyl)phenyl]amino}benzoic acid),
   their derivatives, prodrugs and metabolites.

Typically the microbial infection is caused by one or more strains of H. pylori, such as, for example, the B128, 251 or 26695 strains. In a preferred embodiment the compounds of the present invention show better in vitro activity than observed for commercial BSS and laboratory prepared bismuth salicylate (≧12.5 μg/ml), and for RBC (8 μg/ml) and CBS (≧12.5 μg/ml). In a particularly preferred embodiment the compounds of the present invention have MIC values of 6.25 μg/ml against the three strains of H. pylori (B128, 251 and 26695).

In a fourth aspect of embodiments described herein there is provided a medicament comprising therapeutic amount of a bismuth(III) tris-carboxylate complex having the formula:

[Bi(III)L₃]ₙ

wherein,
L is chosen from the group comprising carboxylato-NSAIDs their derivatives of prodrugs or metabolites,
S is a solvent molecule,
0≦m≧3, and n is ≧1.

In a fifth aspect of embodiments described herein there is provided the use of the bismuth(III) tris-carboxylate complex of the present invention in the preparation of a medicament.

Without wishing to be bound by theory it is believed that the release of ligands (LH) from Bi(III)-L₃ complexes is mediated by hydrolysis of the Bi(III) to decomposition/oxidation products. The hydrolysis is particularly rapid in acidic media such as stomach fluids. Accordingly, the comparatively high stability of the Bi(III) complexes is likely to provide a mechanism for comparatively slow release of NSAIDs from complexes of the present invention. It will be readily apparent to the person skilled in the art that preparations may be suitably formulated to give controlled release of the active compound. For example it may be necessary to combine the bismuth(III) tris-carboxylate complex of the present invention with other excipients, and materials such as enteric coatings to obtain the desired release rate of the NSAID to a subject.

In an alternative or further aspect there is provided a method for the treatment of a human or animal in particular the treatment of conditions characterised by tissue inflammation, comprising administration of an effective amount of a bismuth(III) tris-carboxylate complex according to the present invention.

It is anticipated that compounds of the present application are not only suitable for human medical treatment, but also for animals. A wide range of NSAIDs are used by veterinarians to treat animals. Furthermore, domestic animals such as cats and dogs, and farm animals such as pigs are susceptible to infection by GI bacteria such as H. pylori and a range of other microbes.

### Treatment

In a preferred embodiment the medicament is used for treatment or prevention of pain, fever, inflammation, tissue degeneration or stiffness. In a particularly preferred embodiment the medicament is used for treatment or prevention a disorder chosen from the group comprising: inflammation due to operation or other trauma such as sports injury or burns; gout; hyperuricemia; neuropathic disorders such as post-herpetic neuralgia, trigeminal neuralgia, segmental or intercostals neuralgia, fribromyalgia, causalgia, peripheral neuropathy, diabetic neuropathy, chemotherapy-induced neuropathy, AIDS related neuropathy, occipital neuralgia, geniculate neuralgia, glossopharyngeal neuralgia; toothache; osteo-, rheumatoid or psoriatic arthritis; various forms of headache such as migraine, acute or chronic tension headache, temporomandibular pain, maxillary sinus pain, cluster headache; menstrual cramps, dysmenorrhea; orthostatic hypotension; gastrointestinal disorder; arachnoiditis; spinal disorder such as spinal stenosis, prolapsed disc, sciatica; angina; cancer; cognitive disorders such as dementia including Alzheimer's disease; and Parkinson's disease.

Compounds of the present invention are particularly useful as anti-inflammatory agents. In a preferred embodiment they are used in the treatment of inflammation due to asthma, influenza, chronic bronchitis and rheumatoid arthritis; inflammation in the gastrointestinal tract, such as Crohn's disease ulcerative colitis, inflammatory bowel disease and NSAID induced damage; inflammatory diseases of the skin such as herpes and eczema; inflammatory diseases of the bladder such as cystitis; and eye and dental inflammation.

It will be appreciated that reference to treatment is intended to include prophylaxis as well as the alleviation of established symptoms. Bismuth(III) tris-carboxylate compounds according to the present invention may be administered as the raw chemical but the active species is preferably presented as a pharmaceutical formulation.

Accordingly the invention also provides a pharmaceutical composition which comprises at least one bismuth(III) tris-carboxylate compound or a pharmaceutically acceptable salt or solvate thereof and formulated for administration by any convenient route. Such compositions are preferably in a form adapted for use in medicine, particularly veterinary or human medicine, and can be conveniently formulated in a conventional manner using one or more pharmaceutically acceptable carriers or excipients.

### Dosage Forms

Thus bismuth(III) tris-carboxylate compounds according to the present invention may be formulated for oral, nasal, buccal, parenteral, topical (including ophthalmic and nasal), depot or rectal administration or in a form suitable for administration by inhalation or insufflations (either through the mouth or nose).

For oral administration, the pharmaceutical compositions may take the form of, for example, tablets or capsules prepared by conventional means with pharmaceutically acceptable excipients such as binding agents (eg pregelatinised maize starch, polyvinylpyrrolidone or hydroxypropylmethylcellulose; fillers (eg lactose, microcrystalline cellulose or calcium hydrogen phosphate); lubricants (eg magnesium stearate, talc or silica); disintegrants (eg potato starch or sodium starch glycollate); or wetting agents (eg sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (eg sorbitol syrup, cellulose derivatives or hydrogenated edible fats); emulsifying agents (eg lecithin or acacia); non-aqueous vehicles (eg almond oil, oily esters, ethyl alcohol or fractionated vegetable oils); and preservatives (eg methyl or propyl-p-hydroxybenzoates or sorbic acid). The preparations may also contain buffer salts, flavouring, colouring and sweetening agents as appropriate.

For buccal administration the composition may take the form of tablets or lozenges formulated in a conventional manner.

The compounds of the invention may be formulated for parenteral administration by bolus injection or continuous infusion. Formulations for injection may be presented in unit dosage form (eg in ampoules or in multi-dose containers). The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (eg sterile pyrogen-free water) before use.

The compounds of the invention may be formulated for topical administration in the form of ointments, creams, gels, lotions, pessaries, aerosols or drops (eg eye, ear or nose drops). Ointments and creams may, for example, be formulated with an aqueous or oily base with the addition of suitable thickening and/or gelling agents. Ointments for administration to the eye may be manufactured in a sterile manner using sterilized components. In addition the compounds of the present invention may be incorporated into media such as bandages or wound packings to treat or prevent wound infection by microbes.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of the invention may also be formulated in rectal compositions such as suppositories or retention enemas (eg containing conventional suppository bases such as cocoa butter or other glycerides).

The compounds of the invention may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

The compounds of the invention may also be incorporated into materials used for structural components such as fillings or other packing for teeth or materials used to repair or ‘cement’ breaks in bones.

For intranasal administration, the compounds of the invention may be formulated as solutions for administration via a suitable metered or unitary dose device or alternatively as a powder mix with a suitable carrier for administration using a suitable delivery device.

A proposed dose of the compounds of the invention is from about 1 to about 1000 mg per day. It will be appreciated that it may be necessary to make routine variations to the dosage, depending on the age and condition of the patient and the precise dosage will ultimately be a the discretion of the attendant physical or veterinarian. The appropriate dosage will also depend on the route of administration and the particular compound selected.

Compounds of the present invention may also be used in combination therapy with other pharmaceuticals.

Other aspects and preferred forms are disclosed in the specification and/or defined in the appended claims, forming a part of the description of the invention.

In essence, embodiments of the present invention stem from the realization that gastric injury resulting from use of NSAIDs and concomitant microbial infection can be minimised or inhibited by new, comparatively stable tris-substituted bismuth complexes.

Advantages provided by the present invention comprise the following:
Inhibition or treatment of GI injury such as ulceration and bleeding, including injury caused by short term or long term use of NSAIDs;
Inhibition or treatment of microbial infection such as H. Pylori infection for a patient requiring NSAID administration;
Potential replacement of bismuth subsalicylate, colloidal bismuth subcitrate or ranitidine bismuth citrate in triple and quadruple therapies for H. Pylori treatment when patients require the anti-inflammatory, anti-pyretic or analgesic activity of NSAIDs;
Ease of synthesis, characterisation and manipulation compared to bismuth compounds of the related art;
Stability and comparative lack of hydrolytic sensitivity.

Further scope of applicability of embodiments of the present invention will become apparent from the detailed description given hereinafter. However, it should be understood that the detailed description and specific examples, while indicating preferred embodiments of the invention, are given by way of illustration only, since various changes and modifications within the spirit and scope of the disclosure herein will become apparent to those skilled in the art from this detailed description.

### BRIEF DESCRIPTION OF THE DRAWINGS

Further disclosure, objects, advantages and aspects of preferred and other embodiments of the present application may be better understood by those skilled in the relevant art by reference to the following description of embodiments taken in conjunction with the accompanying drawings, which are given by way of illustration only, and thus are not limitative of the disclosure herein, and in which:

FIG. 1 illustrates some of the carboxylic and benzoic acid NSAIDs suitable for use in the present invention: ketoprofen, naproxen, ibuprofen, mefenamic acid, diflunisal, fenbrufen, sulindac, probenecid, flufenamic acid and tolfenamic acid;

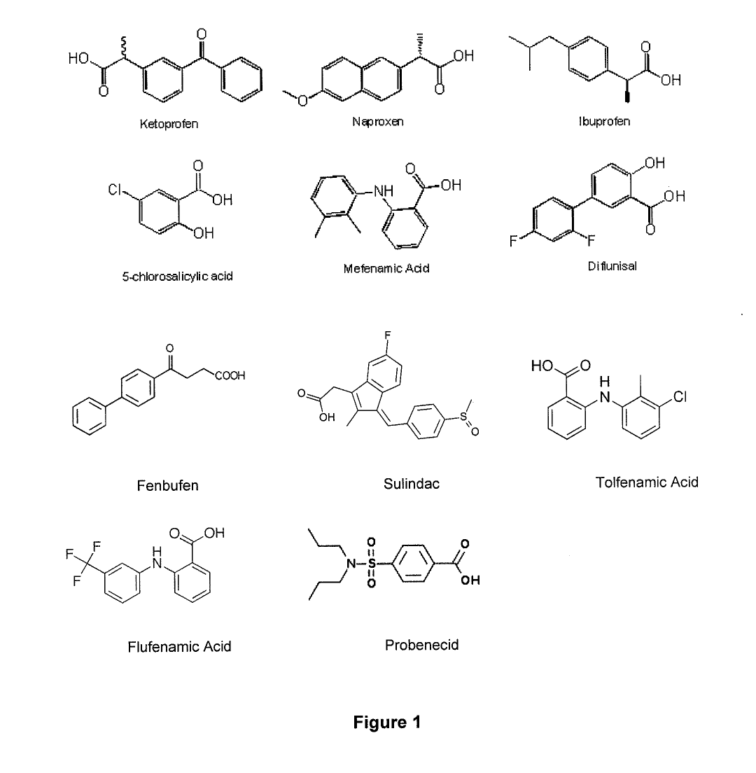

FIG. 2 is a DSC analysis trace of the solvent-free reaction of BiPh₃ with three equivalents of naproxen;

FIG. 3 is a DSC analysis trace of the solvent-free reaction of BiPh₃ with three equivalents of diflunisal; and

FIG. 4 is a DSC analysis trace of the solvent-free reaction of BiPh₃ with three equivalents of ibuprofen.

### DETAILED DESCRIPTION

### Synthesis

To compare the efficacy of the synthetic strategies and to allow for a comparison of their efficiency, reactions to form the fully substituted bismuth(III) tris-carboxylate complexes derived from the free acids of the NSAIDs were conducted on a 1:3 stoichiometric ratio under solvent free conditions (general procedure 2, GP2) and under reflux in toluene (general procedure 1, GP1). The general synthetic procedures are shown in Scheme 1 and described in the Examples.

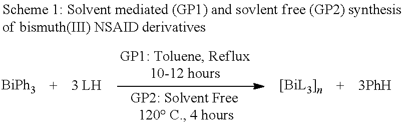

The outcomes of the reactions are shown in Table 1.

TABLE 1
Reaction of the free carboxylic acid form of NSAIDs (=LH)
with BiPh₃ in toluene or ethanol as an inert refluxing solvent.

| | Isolated yield (%) | | | | |
|---|---|---|---|---|---|
| NSAID (=LH) | Reflux | Solvent Free | Formula | Appearance | Mp (° C.) |
| Ketoprofen | 70 | 50 | [BiL₃•H₂O], 1 | Colourless | 52-55 |
| Naproxen | 80 | 80 | BiL₃, 2 | Colourless | 193-194 |
| Ibuprofen | 61 | 90 | BiL₃, 3 | Colourless | 123-125 |
| Mefenamic acid | 82 | 86 | BiL₃, 4 | Yellow | 255-256 |
| Diflunisal | 81 | 82 | BiL₃, 5 | Yellow | 250 |
| 5-chlorosalicylic | 76 | 75 | BiL₃, 6 | Yellow | 245-246 |
| acid | | | | | |
| Fenbufen | 83 | 68 | BiL₃, 7 | Colourless | 206-207 |
| Sulindac | 54 | — | [BiL₃•H₂O], 8 | Yellow | 226-227 |
| Tolfenamic acid | — | 65 | BiL₃, 9 | Yellow | 225-229 |
| Flufenamic acid | 70 | 68 | BiL₃, 10 | Yellow | 208-210 |
| Probenecid | 80 | — | BiL₃, 11 | Colourless | 252-253 |

The data summarised in Table 1 indicates that the isolated yields obtained for complexes 2, 4, 5 and 6 were similar using both synthetic approaches. In the case of 1, however, the isolated yield obtained by the solvent free method is lower. This is mostly due to the fact that the product of the reaction by this method is an oily liquid. After testing a range of solvents and solvent mixtures it was found that the best medium for extracting a solid product was a 1:1 mixture of CH₂Cl₂ and hexane. The reverse was observed for 3. Both methods produce a product which resembles a thick oil or paste. Gentle heating under high vacuum for 2 h leaves a slightly sticky white solid which is soluble in toluene. The higher solubility of 3 in toluene in comparison with the five other compounds accounts for the lower isolated yield from the solvent based reaction since precipitation using methanol is required to obtain a purified solid product.

All the solids 1-11 were assessed for their solubility in common solvents and their hydrolytic stability, and were analysed by NMR, FT-IR, elemental analysis and mass spectrometry. Unfortunately, attempts at obtaining single crystal X-ray structural data proved unsuccessful, mostly due to the small size of the crystals which were obtained. The thermochemical profiles of the solvent free reactions were studied by DSC-TGA.

Compounds 2-6 are sparingly soluble in toluene (3 has the greatest solubility) and acetone and all show greater solubility in DMSO. Compound 1 is soluble in DMSO and CHCl₃. Compounds 7-11 also show greater solubility in DMSO. Compound 9 and 10 is sparingly soluble in toluene as well.

¹H-NMR and ¹³C-NMR spectra for compounds 1-11 were obtained in d₆-DMSO. All the complexes show a general downfield shift for the H and C resonances in comparison to the free acids. This, coupled with the lack of signals corresponding to either the CO₂H proton or residual Ph groups, is good initial evidence for the formation of tris-substituted bismuth(III) carboxylate complexes.

Hydrolysis of bismuth carboxylates leads to the formation of ligand encapsulated oxo-clusters of varying nuclearity, and this can occur either during formation or through subsequent decomposition of the BiL₃ complex.¹¹ If the oxo-cluster species is/are soluble, this is not easily detectable by NMR except through the appearance of signals attributable to the release of free acid. The NMR spectral data on solutions of 1-6 gave no indication of facile hydrolysis and the composition of the solid products as pure [BiL₃]ₙ (or [BiL₃.H₂O]ₙ in the case of 1) was confirmed by elemental analysis.

IR data on 1-6 show a relative shift in the absorbances of the bismuth complexes in comparison to the NSAID free acids. The acids show the stretch corresponding to the carboxyl group in the range 1670-1700 cm⁻¹. All the complexes showed a decrease in wavenumber for the symmetric and asymmetric carboxylate stretches of 35-50 cm⁻¹, indicating deprotonation of the CO₂H group and formation of the carboxylate bound complex. The spectrum of 1 also showed an absorbance band corresponding to OH(H₂O) stretching at 3300 cm⁻¹. In terms of structural information it is interesting to note that for bismuth complexes 1-11 the value of Δv {Δv=vCO₂-(asymm)-vCO₂-(symm)} is less than 200 cm⁻¹, as shown in Table 2. According to Deacon and Phillips since Δv <200 cm⁻¹ then the ligands adopt the bidentate chelating mode.²¹ This is perhaps not unexpected given previous solid state structures of bismuth carboxylates,^{13,14,22-24} the lack of other donor molecules and the high coordination demands of the Bi³⁺ ion.

TABLE 2
IR frequencies of the carboxylate groups (cm⁻¹) observed in the
NSAIDs free acids and their bismuth derivatives.

| | | | | Δv |
|---|---|---|---|---|
| Compound | —CO₂H | —CO₂—_{(asymm)} | —CO₂—_{(symm)} | (cm⁻¹) |
| Ketoprofen | 1689 | — | — | — |
| 1 | — | 1654, 1545 | 1447, 1382 | 175 |
| Naproxen | 1683 | — | — | — |
| 2 | — | 1636, 1522 | 1485, 1393 | 117.5 |
| Ibuprofen | 1721 | — | — | — |
| 3 | — | 1546, 1512 | 1460, 1384 | 107 |
| Mefenamic acid | 1689 | — | — | — |
| 4 | — | 1652, 1580 | 1506, 1372 | 177 |
| Diflunisal | 1684 | — | — | — |
| 5 | — | 1654, 1592 | 1512, 1486 | 124 |
| 5-Chlorosalicylic | 1681 | — | — | — |
| acid | | | | |
| 6 | — | 1665, 1580 | 1524, 1369 | 176 |
| Fenbufen | 1709 | — | — | — |
| 7 | — | 1678, 1560 | 1485, 1354 | 101 |
| Sulindac | 1701 | — | — | — |
| 8 | — | 1654, 1560 | 1466, 1368 | 96 |
| Tolfenamic acid | 1662 | — | — | — |
| 9 | — | 1656, 1582 | 1499, 1372 | 100.5 |
| Flufenamic acid | 1654 | — | — | — |
| 10 | — | 1608, 1508 | 1584, 1399 | 142.5 |
| Probenecid | 1691 | — | — | — |
| 11 | — | 1602, 1522 | 1469, 1346 | 101.5 |

### EXAMPLES

The present invention will be further described with reference to the following non-limiting examples describing the synthesis and characterisation of the compounds of the present invention.

### Materials

BiPh₃ was purchased as a white microcrystalline solid from Strem Chemicals Inc. Ketoprofen, mefenamic acid, diflunisal and 5-sulfosalicylic acid (99%) were purchased from Aldrich Chemical Co. Naproxen was extracted from commercially available Naprogesic (Bayer) tablets.³⁰ The purity was checked by NMR spectroscopy and by melting point analysis. All other reagents and solvents were purchased from Aldrich and used without further purification.

Horse blood agar (HBA) and brain heart infusion broth (BHI) were obtained from Oxoid Australia Pty. Fetal calf serum (FCS) was purchased from Invitrogen. Polymyxin B, Vancomycin, Trimethoprim and Amphotericin B were purchased from Sigma, Mo., USA. Infrared spectra were obtained on a Perkin Elmer 1600 FT-IR. NMR spectra were obtained with Bruker AV-200 and DPX-300 spectrometers with chemical shifts referenced to the appropriate deuterated solvent. Mass spectrometry (ESI) was performed on a Micromass Platform Electrospray Mass Spectrometer. Elemental analysis was carried out by CMAS, Melbourne, Australia and the Campbell Microanalytical Laboratory, New Zealand.

### Bacterial Strains and Culture Conditions

H. pylori strains 251, B128 and 26695³¹ were routinely cultured on HBA or in BHI, supplemented with either 7.5% (v/v) fresh horse blood or 10% (v/v) FCS, respectively.³² Culture media were further supplemented with 155 μg/L polymyxin B, 6.25 mg/L vancomycin, 3.125 mg/L trimethoprim, 1.25 mg/L amphotericin B.

### Determination of the Minimum Inhibitory Concentration (MIC)

The Minimum Inhibitory Concentration (MIC) of the bismuth complexes was determined by the agar dilution technique. For this, H. pylori cultures were incubated in BHI for 18 hours shaking at 140 rpm at 37° C. under microaerobic conditions. Bacteria were pelleted, washed in phosphate-buffered saline, then resuspended in BHI.³¹ Each suspension was adjusted to give an approximate density of 10⁶ bacteria/ml. Aliquots (10 μl) of these suspensions were then streaked onto HBA plates containing doubling dilutions of the different concentrations of bismuth compounds, ranging in concentration from 25 μg/ml to 6.25 μg/ml. Each compound was tested alongside BSS and the NSAIDs, in comparable concentrations. The MICs of the different compounds were determined by examination of the plates after incubation under microaerobic conditions for 3-5 days at 37° C.

### Synthesis of Bismuth(III) NSAID Complexes

### General Procedures

### Solvent Mediated Synthesis: General Procedure 1 (GP1)

For solvent based reactions the reagents were placed in a round bottom flask with toluene (15 ml) and heated to reflux for 10-12 hours. The reaction mixture was allowed to cool to room temperature and the precipitate collected by filtration and washed with a small amount of toluene to remove excess BiPh₃ then dried in air.

### Solvent Free Synthesis: General Procedure 2 (GP2)

For the solvent-free reactions BiPh₃ and the carboxylic acid were ground together and placed in a small pear shaped flask which was heated in an oven at 120° C. for 4 hours. Occasionally the liquid or solid mixture was stirred to assist mixing. After 4 hours the flask was removed from the oven and allowed to cool to room temperature. The solid product was collected and washed with a small amount of toluene to remove excess BiPh₃ then dried in air.

### Example 1

### Reaction of Ketoprofen with BiPh₃ (3:1)

The reaction of BiPh₃ (1.0 mmol, 0.44 g) with ketoprofen (3.0 mmol, 0.77 g) was performed according to GP1. The reaction mixture was homogeneous. On completion of the reaction all volatiles were removed under reduced pressure. A crude solid, which precipitated on removal of the solvent, was taken up in chloroform (5 ml). Addition of hexane (5 ml) precipitated a white solid which was identified as bismuth(III) tris-{2-(3-benzoylphenyl)-propionoate} monohydrate, 1. Yield: 0.68 g, 70%. Melting point: 52-55° C. ¹H NMR (200 MHz, d₆-DMSO) δ=7.40 (27H, m, Ar), 3.86 (3H, q, J=7) ¹³C NMR (50 MHz, d₆-DMSO) δ=194.8 (ArC═OAr) 136.1 (CH), 131.8 (Ar), 131.1 (CH), 128.7 (Ar), 127.7 (Ar), 11.9 (CH₃). m/z (ESI+) 507 [L₂H]⁺, 529 [L₂Na]⁺, 761 [BiL₂(EtOH)]⁺ (ESI−) 785 [BiL₂Cl₂]⁻ (where L=C₁₆H₁₃OCO₂⁻). νₘₐₓ (cm⁻¹) (KBr): 3300 b, 2727 w, 1706 w, 1655 m, 1596 w, 1576 w, 1348 m, 1283 m, 1178 w, 1075 m, 998 w, 953 w, 898 m, 835 m, 774 w, 719 m, 668 m, 667w. Anal. found: C 58.2, H 4.3, Bi 20.8%; BiC₄₈H₄₁O₁₀ requires C 58.4, H 4.1, Bi 21.2%.

The same reaction was conducted using GP2. An oily liquid was obtained on completion of the reaction, which on cooling to room temperature solidified. This solid was taken up in a 1:1 mixture of DCM and hexane (10 ml) and the solution cooled to −20° C. overnight. This produced a microcrystalline precipitate that was collected by filtration and washed with cold ethanol leaving a colourless solid. This was identified as 1 (0.48 g, 50%).

### Example 2

### Reaction of Naproxen with BiPh₃ (3:1)

The reaction of BiPh₃ (1.0 mmol, 0.44 g) with naproxen (3.0 mmol, 0.69 g) was performed and purified according to GP1 and GP2 producing a white solid in both cases. The solid was identified as bismuth(III) tris-{(S)-2-(6-methoxynaphthalen-2-yl)propanote}, 2. Yield: 0.72 g, 80%. Melting point: 193-194° C. ¹H NMR (200 MHz, d₆-DMSO) δ=7.13-7.70 (18H, m, Ar), 3.86 (9H, s, OCH₃), 3.65 (3H, s, CHCH₃), 1.40 (9H, s, CH₃). ¹³C NMR (50 MHz, d₆-DMSO) δ=156.5 (COCH₃), 136.5 (Ar), 132.6 (Ar), 128.5 (Ar), 127.8 (Ar), 126.2 (Ar), 125.9 (Ar), 125.1 (Ar), 117.9 (Ar), 105.2 (Ar), 54.6 (OCH₃), 18.3 (CH₃). m/z (ESI+) 745.2 [BiL₂(DMSO)]⁺, 823 [BiL₂(DMSO)₂]⁺; (ESI−) 229 [L]⁻, 459 [L₂]⁻ (where L=C₁₃H₁₃OCO₂⁻). νₘₐₓ (cm⁻¹) (KBr): 3447 m, 2930 w, 1636 s, 1606 s, 1560 w, 1522 w, 1507 s, 1485 m, 1458 m, 1393 vs, 1273 vs, 1230 s, 1215 s, 1160 m, 1073 w, 1031 s, 928 w, 853 s, 809 m, 751 w, 713 m, 670 w, 473 s. Anal. found; C 56.1, H 4.4, Bi 22.9%; BiC₄₂H₃₉O₉ requires C 56.3, H 4.4, Bi 23.3%.

### Example 3

### Reaction of Ibuprofen with BiPh₃ (3:1)

The reaction of BiPh₃ (1.0 mmol, 0.44 g) with ibuprofen (3.0 mmol, 0.62 g) was performed according to GP1. On cooling to room temperature the final reaction mixture is a homogeneous toluene solution. All volatiles were removed under reduced pressure leaving a thick oily colourless liquid. Addition of methanol (10 ml) precipitated a white solid which was identified as bismuth(III) tris-{2-(4-isobutylphenyl)-propionoate}, 3.

Yield: 0.50 g, 60.6%. Melting point: 123-125° C. ¹H NMR (300 MHz, d₆-DMSO) δ=7.18 (6H, d, J=7.7 Hz, Ar), 7.05 (6H, d, J=7.7 Hz, Ar), 3.49 (3H, q, CH), 2.40 (6H, d, J=7.0 Hz, CH₂), 1.80 (3H, m, CH₂CH), 1.15 (18H, d, J=6.9 Hz, CH₃), 0.84 (9H, d, J=6.6 Hz, CH₃). ¹³C NMR (75 MHz, d₆-DMSO) δ=170.6 (COOBi), 138.6 (CCH₂), 128.1 (Ar), 126.7 (Ar), 43.7 (CH₂), 29.0 (C(H)Me), 28.4 (CH(CH₃)₂), 21.6 (CH₃), 18.4 (CH₃). m/z (ESI+) 229 [LHNa]⁺, 847 [BiL₃Na]⁺; (ESI−) 205 [L]⁻, 773 [BiL₂{(DMSO)-H}₂]⁻ (where L=C₁₂H₁₇CO₂⁻). νₘₐₓ (cm⁻¹) (KBr): 2955 m, 2868 w, 1546 m, 1512 m, 1460 m, 1384 s, 1358 w, 1261 m, 1168 m, 1067 w, 1022 w, 893 w, 800 w. Anal. Found; C 55.1, H 6.3%; BiC₃₉H₅₁O₆ requires C 56.8, H 6.2%.

In a variation to GP2 the reaction mixture was heated to 85° C. for 2 h. On cooling a thick colourless gel was obtained. The sample was then dried under high vacuum for 2 h leaving a sticky white solid. This solid, which is soluble in toluene, analysed as compound 3. Yield: 0.74 g, 90%.

### Example 4

### Reaction of Mefenamic Acid with BiPh₃ (3:1)

Reaction of BiPh₃ (1.0 mmol, 0.44 g) with mefenamic acid (3.0 mmol, 0.72 g) was performed and purified according to GP1 and GP2, producing a yellow solid. This was identified as bismuth(III) tris-{2-(2,3-dimethylphenyl)aminobenzoate}, 4. Yield: 0.76 g, 82% (GP1); 0.80 g, 86% (GP2). Melting point: 255-256° C. ¹H NMR (200 MHz, d₆-DMSO) δ=9.47 (3H, s, NH), 7.85 (3H, d, J=6.94 Hz, Ar), 7.25 (3H, m, Ar), 7.03 (6H, m, Ar), 6.80 (9H, m, Ar), 2.15 (9H, s, CH₃), 1.93 (s, 9H, CH₃). ¹³C NMR (50 MHz, d₆-DMSO) δ=142.1 (COOBi), 133.3 (C—CH₃), 132.1 (C—NH), 128.0 (Ar), 126.5 (C—CH₃), 124.6 (Ar), 123.4 (Ar), 122.7 (Ar), 120.1 (Ar), 115.1 (Ar), 110.8 (Ar), 107.8 (C—COO—Bi), 14.7 (CH₃), 7.85 (CH₃). m/z (ESI+) 604 [BiL(DMSO)₂]⁺; (ESI−) 985.5 [BiL₃Cl(H₂O)]⁻ (where LH═C₁₄H₁₄NCO₂⁻, L=C₁₄H₁₃NCO₂²⁻). νₘₐₓ (cm⁻¹) (KBr): 2361 m, 1652 s, 1614 m, 1580 vs, 1506 s, 1472 w, 1454 w, 1372 m, 1262 s, 1161 w, 1096 w, 866 w, 778 w, 752 w, 681 vw. Anal. found; C, 58.3; H, 4.6; N, 4.5, Bi 22.5%; BiC₄₅H₄₂N₃O₆ requires C, 58.1; H, 4.6; N, 4.5, Bi 22.5%.

### Example 5

### Reaction of Diflunisal with BiPh₃ (3:1)

The reaction of BiPh₃ (1.0 mmol, 0.44 g) with diflunisal (3.0 mmol, 0.75 g) was performed and purified according to GP1 and GP2. This produced a yellow solid which was identified as bismuth(III) tris-{2′,4′-difluoro-4-hydroxybiphenyl-3-carboxylate}, 5.

Yield: 0.77 g, 81% (GP1), 0.78 g, 82% (GP2). Melting point: decomposes above 250° C. ¹H NMR (200 MHz, d₆-DMSO) δ=7.88 (3H, s, Ar), 7.55-6.78 (15H, m, Ar). ¹³C NMR (50 MHz, d₆-DMSO) δ=156.8 (C—F), 130.8 (Ar), 128.9 (Ar), 127.7 (Ar), 125.5 (Ar), 123.7 (C—OH), 111.3 (Ar), 103.8 (Ar). m/z (ESI+) 785 [BiL₂(DMSO)]⁺, 863 [BiL₂(DMSO)₂]⁺; (ESI−) 249 [L]⁻. νₘₐₓ (cm⁻¹) (KBr): 3154 w, 2925 w, 1654 s, 1618 w, 1592 w, 1512 w, 1486 m, 1453 m, 1268 w, 1239 w, 1214 w, 1144 m, 1106 w, 1096 m, 1039 w, 967 m, 852 vw, 837 s, 812 w, 792 vw, 727 vw, 661 s. Anal. found; C 48.9, H 2.2, F 11.6, Bi 21.5%; BiC₃₉H₂₁F₆O₉ requires C 48.9, H 2.3, F 11.9, Bi 21.9%

### Example 6

### Reaction of 5-chlorosalicylic Acid with BiPh₃ (3:1)

The reaction of BiPh₃ (1.0 mmol, 0.44 g) with 5-chlorosalicylic acid (3.0 mmol, 0.52 g) was performed and purified according to GP1 and GP2. This produced a yellow solid which was identified as bismuth(III) tris-{5-chlorosalicylate}, 6. Yield: 0.55 g, 76% (GP1); 0.54 g, 75% (GP2). Melting Point: 245-246° C. ¹H NMR (300 MHz, d₆-DMSO) δ=□7.70 (s, 3H, Ar), 6.83 (d, 3H, J=8.1 Hz, Ar), 6.79 (s, 3H, Ar). ¹³C NMR (75 MHz, d₆-DMSO) δ=159.3 (C—OH), 132.2 (Ar), 128.6 (Ar) 127.9 (C—Cl), 127.2 (Ar), 115.8 (C—COO). νₘₐₓ (cm⁻¹) (KBr): 1665 m, 1621 w, 1580 w, 1567 m, 1524 m, 1471 m, 1413 m, 1369 w, 1287 m, 1234 w, 1211m, 1107 w, 1074 w, 892 m, 827 m, 794 w, 722 s, 694 vw, 650 m. H 1.67%, Bi 28.8%. m/z (ESI+) 535 [Bi(L)]⁺, 629 [Bi(LH)₂(DMSO)]⁺, 707 [Bi(LH)₂(DMSO)₂]⁺; (ESI−) 549 [Bi(L)₂]⁻, 723 [Bi(LH)₂{(DMSO)-H}₂H₂O]⁻. Anal. found: C 34.1, H 1.9, Bi 27.9%; BiC₂₁H₁₂Cl₃O₉ requires C 34.8, H 1.7, Bi 28.8%.

### Example 7

### Reaction of Fenbufen with BiPh₃ (3:1)

Reaction of BiPh₃ (1.0 mmol, 0.44 g) with fenbufen (3.0 mmol, 0.76 g) was performed and purified according to GP1 and GP2, producing a white solid. This was identified as bismuth(III) tris-{γ-oxo-(1,1′-biphenyl)-4-butanoate}, 7. Yield: 0.97 g, 83% (GP1); 0.66 g, 68% (GP2). Melting point: 206-207° C. ¹H NMR (200 MHz, d₆-DMSO) δ=8.04 (6H, d, J=8.0 Hz, Ar), 7.80 (6H, d, J=8.0 Hz, Ar), 7.72 (6H, d, J=8.0 Hz, Ar), 7.44 (9H, m, Ar), 3.36 (6H, t, CH₂), 2.58 (6H, t, CH₂). ¹³C NMR (50 MHz, d₆-DMSO) δ=197.7 (ArCOCH₂), 173.6 (COOBi), 144.1 (Ar), 138.5 (Ar), 134.9 (Ar), 128.6 (Ar), 128.0 (Ar), 126.5 (Ar), 32.8 (ArCOCH₂CH₂), 27.7 (ArCOCH₂CH₂). νₘₐₓ (cm⁻¹) (KBr): 1678 s, 1603 m, 1560 w, 1485 w, 1401 m, 1354 m, 1249 w, 1196 m, 982 m, 838 m, 765 s, 720 w, 690 s, 627 w. Anal. found; C 60.6, H 4.3%; BiC₄₈H₃O₉ requires C 59.5, H 4.1%.

### Example 8

### Reaction of Sulindac with BiPh₃ (3:1)

The reaction of BiPh₃ (0.3 mmol, 0.14 g) with sulindac (1 mmol, 0.36 g) was performed in ethanol (heated to reflux for 6-7 hours). On cooling to room temperature the final reaction mixture was a homogeneous ethanol solution. All volatiles were removed under reduced pressure leaving a thick oily yellow liquid. Addition of methanol (5 ml) precipitated a white solid which was identified as bismuth(III) tris-{(Z)-2-(5-fluoro-2-methyl-1-(4-(methylsulfinyl)benzylidene)-1H-inden-3-yl)acetate} monohydrate, 8.

Yield: 0.22 g, 54% (GP1). Melting point: 226-227° C. ¹H NMR (200 MHz, d₆-DMSO) δ=7.78 (6H, d, J=8.4 Hz, Ar), 7.70 (6H, s, J=8.2 Hz, Ar), 7.30 (3H, s, ArCH=Cp), 7.15 (3H, dd, J=8.3 Hz, Ar), 7.00 (3H, dd, J=8.0 Hz, Ar), 6.68 (3H, m, Ar), 3.50 (6H, s, CH₂), 2.81 (9H, s, SOCH₃), 2.14 (9H, s, CH₃). ¹³C NMR (50 MHz, d₆-DMSO) δ=165.4 (COOBi), 160.6 (CF), 147.6 (Cp), 146.7 (CSOCH₃), 140.9 (Cp), 139.0 (Ar), 138.2 (C—CH₃), 130.4 (Cp), 129.9 (Cp), 129.8 (Cp) 124.4 (Ar), 123.7 (Ar), 123.5 (Ar), 110.8 (Ar), 106.6 (Ar), 43.6 (SOCH₃) 19.0 (CH₂), 10.7 (CH₃). m/z (ESI+) 377 [LNa]⁺, 1275 [BiL₃H]⁺ (where L=C₁₉H₁₆FOSCO₂⁻). νₘₐₓ (cm⁻¹) (KBr): 3448 b, 1654 w, 1603 m, 1560 m, 1466 s, 1368 m, 1262 w, 1195 w, 1166 m, 1086 m, 997 w, 915 m, 891 m, 856 m, 811 m, 728 m, 680 m, 657 w. Anal. found; C 55.3, H 3.8%; BiC₆₀H₅₀F₃O₁₀S₃ requires C 55.7, H 3.9%.

### Example 9

### Reaction of Tolfenamic Acid with BiPh₃ (3:1)

Reaction of BiPh₃ (1.0 mmol, 0.44 g) with tolfenamic acid (3.0 mmol, 0.78 g) was performed and purified according to GP2, producing a yellow solid. This was identified as bismuth(III) tris-{2-(3-chloro-2-methylphenyl)aminobenzoate}, 9. Yield: 0.64 g, 65%. Melting point: 225-229° C. ¹H NMR (400 MHz, d₆-DMSO) δ=9.70 (3H, s, NH), 7.87 (3H, d, J=7.2 Hz, Ar), 7.34-7.17 (12H, br, Ar), 6.95 (3H, br, Ar) 6.81 (3H, br, Ar), 2.07 (9H, s, CH₃). ¹³C NMR (100 MHz, d₆-DMSO) δ=146.1 (C—NH), 141.0 (C—NH), 137.0 (C—Cl), 136.9 (Ar), 134.2 (Ar) 132.5 (C—CH₃), 131.9 (Ar), 128.1 (Ar), 123.6 (Ar), 119.7 (Ar), 117.5 (Ar), 113.9 (C—COOBi), 14.7 (CH₃). m/z (ESI−) 604 [BiL₂Cl₂]⁻, 1081.6 [BiL₃Cl(H₂O)₃]⁻ (where L=C₁₃H₁₁ClNCO₂⁻). νₘₐₓ (cm⁻¹) (KBr): 1656 s, 1612 m, 1582 vs, 1499 s, 1453 m, 1372 m, 1262 s, 1276 s, 1162 w, 1011 m, 854 m, 778 s, 751 s, 669 m. Anal. found; C, 51.4; H, 3.6; N, 4.1%; BiC₄₂H₃₃Cl₃N₃O₆ requires C, 50.9; H, 3.4; N, 4.2%.

### Example 10

### Reaction of Flufenamic Acid with BiPh₃ (3:1)

Reaction of BiPh₃ (1.0 mmol, 0.44 g) with flufenamic acid (3.0 mmol, 0.84 g) was performed and purified according to GP1 and GP2, producing a yellow solid. This was identified as bismuth(III) tris-{2-(3-(trisfluoromethyl)phenyl)aminobenzoate}, 10. Yield: 0.71 g, 70% (GP1); 0.69 g, 68% (GP2). Melting point: 208-210° C. ¹H NMR (400 MHz, d₆-DMSO) δ=10.0 (3H, s, NH), 7.99 (3H, d, J=6.4 Hz, Ar), 7.31 (18H, m, Ar), 6.92 (3H, m, Ar). ¹³C NMR (100 MHz, d₆-DMSO) δ=173.4 (COOBi), 145.2 (C—NH), 142.9 (C—NH), 137.8 (Ar), 133.9 (Ar), 132.7 (Ar), 131.2 (Ar) 130.6 (C—CF₃), 126.5 (CF₃), 123.5 (Ar), 122.8 (CCOOBi), 119.4 (Ar), 118.6 (Ar), 116.2 (Ar) 115.5 (Ar). m/z (ESI+) 847 [BiL₂(DMSO)]⁺, 925 [BiL₂(DMSO)₂]⁺, 1150 [BiL₃Na(DMSO)]⁺ (where L=C₁₃H₉F₃NCO₂⁻). νₘₐₓ (cm⁻¹) (KBr): 1584 m, 1508 m, 1465 m, 1399 m, 1333 s, 1275 m, 1162 m, 1122 m, 1069 w, 930 m, 865 m, 791 m, 747 s 696 s, 664 m. Anal. found; C, 48.1; H, 2.5; N, 3.9%; BiC₄₂H₂₇F₉N₃O₆ requires C, 48.1; H, 2.6; N, 4.0%.

### Example 11

### Reaction of Probenecid with BiPh₃ (3:1)

Reaction of BiPh₃ (1.0 mmol, 0.44 g) with probenecid acid (3.0 mmol, 0.86 g) was performed in ethanol (heated to reflux for 6-7 hours). The reaction mixture was allowed to cool to room temperature and the precipitate collected by filtration and washed with a small amount of ethanol to remove excess BiPh₃ then dried in air. The product was identified as bismuth(III) tris-{4-(dipropylsulfamoyl)benzoate}, 11. Yield: 0.85 g, 80%. Melting point: 225-226° C. ¹H NMR (200 MHz, d₆-DMSO) δ=8.13 (6H, d, J=6.8 Hz, Ar). 7.87 (6H, d, J=6.8 Hz, Ar), 3.05 (12H, t, CH₂), 1.44 (12H, q, CH₂), 0.79 (18H, t, CH₃). ¹³C NMR (50 MHz, d₆-DMSO) δ=168.7 (COOBi), 145.0 (CSO₂), 132.0 (C—COOBi), 132.0 (Ar), 128.8 (Ar), 51.4 (CH₂), 23.4 (CH₂), 12.7 (CH₃). m/z (ESI+) 1163 [BiL₃Na(DMSO)]⁺; (ESI−) 1138 [BiL₃{(DMSO)—H}]⁻ (where L=C₁₃H₁₈NO₂SCO₂⁻). νₘₐₓ (cm⁻¹) (KBr): 2966 m, 1602 m, 1574 m, 1522 m, 1469 m, 1427 m, 1346 s, 1288 s, 1158 s, 1084 s, 997 m, 985 m, 930 m, 866 s, 797 s, 778 m, 765 m, 737 s, 711 w, 688 m. Anal. found; C, 44.1; H, 5.4; N, 3.8%; BiC₃₉H₅₄N₃O₁₂S₃ requires C, 44.1; H, 5.1; N, 3.9%.

### Example 12

### DSC-TGA Studies

All the solvent free reactions were studied using differential scanning calorimetry (DSC) and thermogravimetric analysis (TGA). The success of the solvent free reactions can be observed in the DSC profiles of the reaction mixtures. The energy changes relating to the temperature and phase dependent reaction of the acid with BiPh₃ observed in the DSC can be correlated directly with the loss of PhH observed in the TGA trace. Final and rapid decomposition of the BiL₃ compounds derived from naproxen, mefenamic acid and diflunisal are observed at 292, 260 and 250° C. respectively. The product from the reaction with ibuprofen undergoes two distinct exothermic decomposition processes at 270 and 300° C. while the product from 5-chlorosalicylic acid does not appear to decompose below 350° C. In general, this is in agreement with reports on the thermal decomposition of bismuth medicinals, corresponding closely to a ‘stage III’ decomposition process involving decarboxylation and combustion of organic components.²⁵

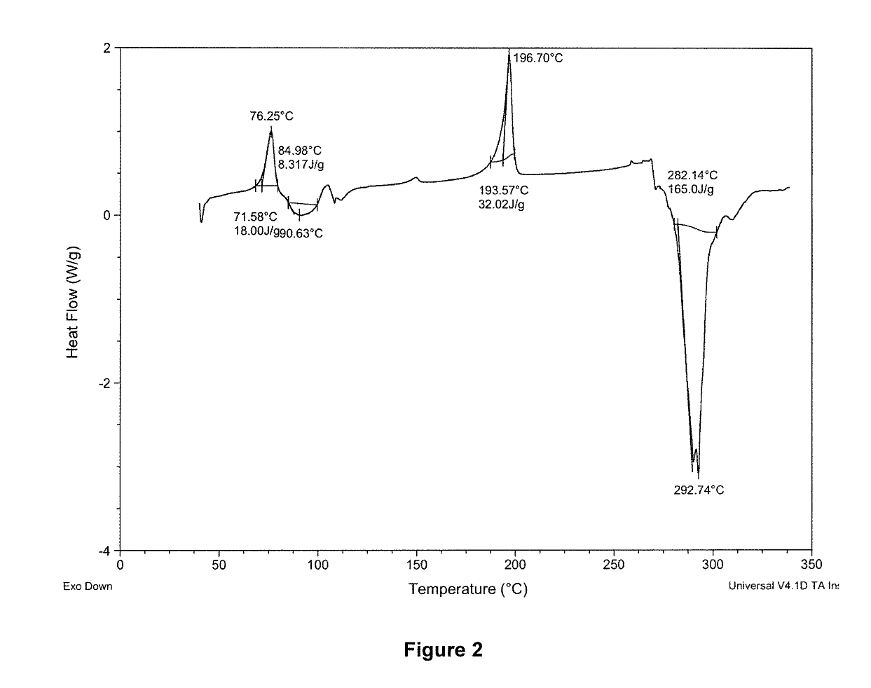

As an illustrative example, FIG. 2 shows the DSC trace for the reaction of naproxen with BiPh₃ in a 3:1 stoichiometric ratio. The endotherm at 76.2° C. corresponds to the melting of BiPh₃. As is now typical for these systems, at the onset of melting the reaction begins and an exotherm is observed (ca 85° C.). From the TGA this corresponds to the loss of 3 PhH. The beginning of the endotherm at 193° C. and centred at 196° C. corresponds with the melting point of the tris-substituted product, consistent with that found experimentally (193-194° C.). The final large exotherm at 292° C. indicates rapid decomposition of BiL₃.

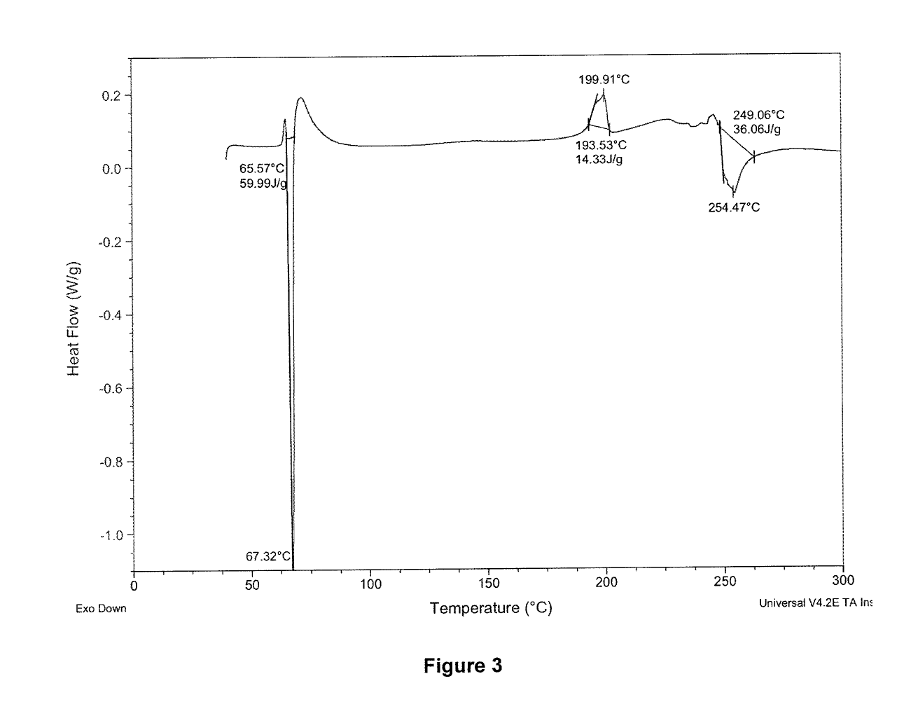

The DSC profile for the reaction of diflunisal with BiPh₃ displays a distinct and unusual feature in comparison with the other reactions performed herein. As can be seen in FIG. 3, the exotherm at 65.5° C. suggests that the onset of the reaction occurs before BiPh₃ is expected to begin melting at ca 78° C. (diflunisal melts at 211-213° C.). This exotherm corresponds in the TGA to a loss of three equivalents of PhH, indicative of a complete reaction. One plausible reason for the unusual observation of the reaction occurring prior to generation of a partial melt phase could be the fact that the presence of fluorine in the ligand, an electron withdrawing group, stabilises the carboxylate anion and increases the acidity of the molecule, making it more reactive.

This pattern of reactivity can be observed experimentally on a larger scale. When the solvent-free reaction of diflunisal with BiPh₃ was conducted in an oil bath maintained at 70° C. a gradual colour change from colourless to yellow was observed as the reaction proceeded. The formation of a ‘reactive’ liquid phase was not visible by the naked eye. Analysis of the final product 4 is consistent with that obtained from the solvent free reaction conducted at 120° C., and that obtained by reflux in toluene.

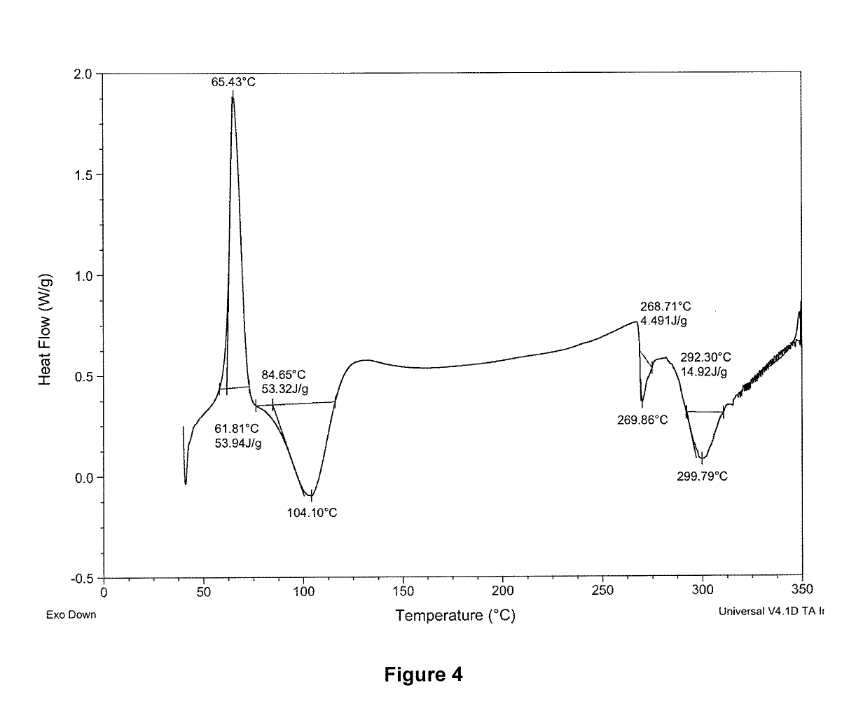

The low melting point of pure ibuprofen (76° C.) means the reactive melt phase with BiPh₃ occurs at the relatively low temperature of 65° C. This depressed melting point for the mixture is lower than that of both reactants. The DSC trace, FIG. 4, indicates that the reaction to form the tris-substituted product occurs rapidly thereafter. Therefore the solvent free reaction was conducted at 85° C. for only 2 h, leading to near quantitative formation of 3. If the standard GP2 conditions are employed (ie 4 h) there is clear evidence of partial decomposition as the liquid phase blackens and an impure solid is obtained on cooling and extraction.

### Example 13

### Stability Studies

To assess the compounds for stability to atmospheric moisture, microanalytical data was collected on a regular basis over a period of six months. During this time there was no appreciable change in the mass % of C, H and N indicating that the compounds are air stable over that time, or hydrolyse only very slowly. The compounds were also suspended in deionised water and stirred overnight before being collected by filtration and dried. Melting point analyses indicted little or no decomposition. The compounds also appear to be stable in solution. NMR spectra on the compounds were recorded in C₆D₆ before and after the addition of two drops of D₂O. There were no observable changes in the chemical shift patterns.

The underlying premise of generating the bismuth derivatives of NSAIDs is that on oral administration the complexes decompose in the acidic environment of the stomach, thereby producing bismuth salts capable of assisting GI protection and healing, and killing H. pylori. This would be accompanied by liberation of the NSAID, predominantly in its free acid form (LH), which on absorption would provide their typical and expected therapeutic effects.

To assess this, compounds 1-6 were added to a 1M solution of HCl. After 10-15 mins any free organic acid generated was extracted into diethyl ether, dried and analysed by NMR, and purity confirmed by melting point analysis. It was found that for all complexes the NSAIDs in their free acid form were gradually released, and could ultimately be extracted from HCl solution in near quantitative amounts while Bi³⁺ remained soluble (presumably as a solution of BiOCl in HCl(aq)). Dependent on pH and the contents of the stomach, in vivo bismuth can remain bound to ionised carboxylate, form complexes with other intestinal anions (CI, citrate) and also bind with sulfur rich proteins.²⁶

### Example 14

### Activity against H. pylori

An assessment of the antibacterial activity of complexes 1-6, bismuth tris-salicylate [Bi(Hsal)₃]ₙ bismuth subsalicylate (BSS), and the NSAIDs in their free acid form (LH) was carried out against three standard laboratory strains of H. pylori: B128, 251 and 26695, using compound concentrations ranging from 2 to 0.0625 mg ml⁻¹. The Minimum Inhibitory Concentration (MIC) of each was determined by the Agar Diffusion method (as described above). The activity of all complexes was found to be ≧6.25 μg/ml; below this confluent growth of the bacteria was observed. These activities compare favourably with those of the control compounds: bismuth salicylate, as prepared in a laboratory, and the commercial sample of BSS were active at ≧12.5 μg/ml, while the NSAIDs in their free acid form were inactive (>25 μg/ml). The MIC value of these complexes is lower than for RBC (8 μg/ml) and for CBS (≧12.5 μg/ml) indicating they display better in-vitro anti-bacterial activity. They also compare well with the activity of other bismuth compounds previously reported in the literature (4-64 μg/ml).²⁷⁻²⁹

These data suggest that bismuth complexes 1-11 are more effective than current standard bismuth preparations at killing H. pylori strains. One contributing factor to this may be the lack of prior hydrolysis and decomposition meaning the bismuth compounds are applied in their pure BiL₃ form rather than in an oxidised ‘sub’ form.

While this invention has been described in connection with specific embodiments thereof, it will be understood that it is capable of further modification(s). This application is intended to cover any variations uses or adaptations of the invention following in general, the principles of the invention and including such departures from the present disclosure as come within known or customary practice within the art to which the invention pertains and as may be applied to the essential features hereinbefore set forth.

As the present invention may be embodied in several forms without departing from the spirit of the essential characteristics of the invention, it should be understood that the above described embodiments are not to limit the present invention unless otherwise specified, but rather should be construed broadly within the spirit and scope of the invention as defined in the appended claims. The described embodiments are to be considered in all respects as illustrative only and not restrictive.

Various modifications and equivalent arrangements are intended to be included within the spirit and scope of the invention and appended claims. Therefore, the specific embodiments are to be understood to be illustrative of the many ways in which the principles of the present invention may be practiced. In the following claims, means-plus-function clauses are intended to cover structures as performing the defined function and not only structural equivalents, but also equivalent structures.

“Comprises/comprising” and “includes/including” when used in this specification is taken to specify the presence of stated features, integers, steps or components but does not preclude the presence or addition of one or more other features, integers, steps, components or groups thereof. Thus, unless the context clearly requires otherwise, throughout the description and the claims, the words ‘comprise’, ‘comprising’, ‘includes’, ‘including’ and the like are to be construed in an inclusive sense as opposed to an exclusive or exhaustive sense; that is to say, in the sense of “including, but not limited to”.

### REFERENCES

1. G. Treiber, P. Malfertheiner and U. Klotz, Expert Opin. Pharmacother., 2007, 8, 329.
2. S. Marchi, F. Costa, F, M. Bellini, C. Belcari, M. G. Mumolo, R. Tornar, R. Spisni, E. Torel and G. Maltinti, Eur. J. Gastroenterol. Hepatol., 2002, 13, 547.
3. J. Houghton, J. G. Fox and T. C. Wang, Eur. J. Gastroenterol. Hepatol., 2002, 17, 495.
4. J. R. Lambert and P. Midolo, Aliment. Pharmacol. Ther., 1997, S11, 27.
5. U. Dittes, E. Vogel and B. K. Keppler, Coord. Chem. Rev., 1997, 163, 345.
6. J. P. Gisbert, World J. Gastroenterol., 2008, 14, 5385.
7. N. Yang and H. Sun, Coord. Chem. Rev., 2007, 251, 2354
8. H. P. Rang, M. M. Dale, J. M. Ritter and J. Flower, Pharmacology, 6^{th} Edn., Elsevier, UK, 2007.
9. M. M. Wolf, D. R. Lichtenstein and G. Singh, N. Engl. J. Med., 1999, 340, 1888.
10. D. Schaffer, T. Florin, C. Eagle, I. Marschner, G. Singh, M. Grobler, C. Fenn, M. Schou and K. M. Curnow, Med. J. Aust. (MJA) 2006, 185, 501.
11. M. Mehring, Coord. Chem. Rev., 2007, 251, 974.
12. G. G. Briand and N. Burford, Chem. Rev., 1999, 99, 2601.
13. P. C. Andrews, G. B. Deacon, W. R. Jackson, M. Maguire, N. M. Scott, B. W. Skelton and A. H. White, J. Chem. Soc., Dalton Trans., 2002, 4634.
14. P. C. Andrews, G. B. Deacon, P. C. Junk, I. Kumar and M. Silberstein, 2006, Dalton Trans., 2006, 4852.
15. P. C. Andrews, G. B. Deacon, P. C. Junk, I. Kumar, and J. G. MacLellan, Organometallics, 2009, 28, 3999.
16. P. C. Andrews, G. B. Deacon, C. M. Forsyth, P. C. Junk, I. Kumar and M. Maguire, Angew. Chem. Int. Ed., 2006, 45, 5638.
17. W. Diamantis, J. Melton, R. D. Sofia and V. B. Ciofalo, Toxicol. Appl. Pharmacol., 1980, 52, 454.
18. I. Turel, L. Golic, P. Bukovec and M. Gubina, 1998, J. Inorg. Biochem., 1998, 71, 53.
19. A. R. Shaikh, R. Giridhar and M. R. Yadav, Int. J. Pharm., 2007, 332, 24.
20. M. Abuznaid, A.-S. Sallam, I. Hamdan, M. Al-Hussaini and A. Bani-Jaber, Drug Dev. Ind. Pharm., 2008, 34, 434.
21. G. B. Deacon and R. J. Phillips, Coord. Chem. Rev., 1980, 33, 227.
22. A. D. Rae, G. J. Gainsford and T. Kemmit, Acta Cryst., 1998, B54, 438.
23. V. V. Sharutin, I. V. Egorova and T. K. Ivanenko, Rus. J. Coord. Chem., 2004, 30, 309.
24. S. I. Troyanov, A. P. Pisarevsky, J. Chem. Soc. Chem. Commun., 1993, 335.
25. A. Radecki and M. Wesolowski, Thermochim. Acta, 1976, 17, 217
26. J. R. Lambert and P. Midolo, Aliment. Pharmacol. Ther., 1997, 11, 27.
27. C. A. McNulty, J. Dent and R. Wise, Antimicrob. Agents Chemother., 1985, 28, 837.
28. C. E. Haas, D. E. Nix and J. J. Schentag, Antimicrob. Agents Chemother., 1990, 34, 1637.
29. Y. Glupczynski, M. Delmee, C. Bruck, M. Labbe, V. Avesani and A. Burette, Eur. J. Epidemiol., 1988, 4, 154.
30. T. D. Walsh and C. S. Koontz, J. Chem. Educ. 1997, 74, 585.
31. J. Viala, I. G. Boneca, A. Cardona, S. E. Girardin, A. P. Moran, R. Athman, S. Mémet, M. R. Huerre, A. J. Coyle, P. S. DiStefano, P. J. Sansonetti, A. Labigne, J. Bertin, D. J. Philpott and R. L. Ferrero, Nat. Immunol., 2004, 5, 1166.
32. R. L. Ferrero, J.-M. Thiberge, M. Huerre and A. Labigne, Infect. Immun., 1998, 66, 1349.

1. A bismuth(III) tris-carboxylate complex of formula:
[Bi(III)L₃]ₙ
and its pharmaceutically acceptable salts and solvates,
wherein,
L is chosen from the group comprising carboxylato-NSAIDs, their derivatives, prodrugs or metabolytes, and
n is ≧1.

2. A compound according to claim 1 wherein the bismuth(III) tris-carboxylate complex is a solvate of formula, [Bi(III)L₃.Sₘ]ₙ wherein S is the solvate molecule, and m is ≧1.

3. A compound according to any one of claim 1 or claim 2 wherein the carboxylato-NSAID is chosen from the group comprising ketoprofen, naproxen, ibuprofen, 5-chlorosalicylic acid, mefenamic acid, diflunisal, fenbrufen, sulindac, probenecid, tolfenamic acid, flufenamic acid, meseclozone, their derivatives and metabolytes.

4. A compound according to claim 2 wherein L is chosen from ketoprofen or sulindac.

5. A compound according to claim 1 wherein L is a metabolite of Meseclozone.

6. A compound according to claim 1 or claim 2 having MIC values of ≧6.25 μg/ml against a strains of H. pylori chosen from the group comprising B128, 251 and 26695.

7. A method of producing a bismuth(III) tris-carboxylate complex of formula: [Bi(III) L₃]ₙ
its pharmaceutically acceptable salts and solvates,
wherein,
L is chosen from the group comprising carboxylato-NSAIDs their derivatives, prodrugs or metabolytes,
n is ≧1,
the method comprising the step of reacting a Bi(III) compound with LH.

8. A method according to claim 7 wherein the Bi(III) compound is BiPh₃ and the ratio of Bi(III) to LH is 1:≧3.

9. A method of treating microbial infection including the step of administering to a subject a therapeutic amount of a bismuth(III) tris-carboxylate complex having the formula:
[Bi(III)L₃Sₘ]ₙ
wherein,
L is chosen from the group comprising carboxylato-NSAIDs their derivatives prodrugs or metabolites,
S is a solvent molecule,
0≦m≧3, and n is ≧1.

10. A method according to claim 9 wherein the carboxylato-NSAID is chosen from the group comprising ketoprofen, naproxen, ibuprofen, 5-chlorosalicylic acid, mefenamic acid, diflunisal, fenbrufen, sulindac, probenecid, tolfenamic acid, flufenamic acid, meseclozone, their derivatives and metabolites.

11. A method according to claim 9 or claim 10 wherein the microbial infection is caused by bacteria chosen from the group comprising Helicobacter pylori, Escherichia coli, Klebsiella pneumoniae, Bacillus pumilus, Staphylococcus aureus and Staphylococcus epidermidis.

12. The use of a bismuth(III) tris-carboxylate complex according to claim 1 or claim 2 for the preparation of a medicament formulated for an administrative route chosen from oral, nasal, buccal, parenteral, topical, depot or rectal.

13. A method for the treatment of a human or animal subject comprising the step of administering an effective amount of a bismuth(III) tris-carboxylate complex or a pharmaceutically acceptable salt or solvate thereof.

14. A method according to claim 13 for the treatment or prevention of a disorder chosen from the group comprising pain, fever, inflammation, tissue degeneration, stiffness or combinations thereof.
